(19) United States
(12) Patent Application Publication
(10) Pub. No.: US 2010/0323409 A1
(43) Pub. Date: Dec. 23, 2010

(54) PROCESS FOR PRODUCING (2S,3R,4S)-4-HYDROXY-L-ISOLEUCINE

(76) Inventors: Sergey Vasilievich Smirnov, Moscow (RU); Natalia Nikolaevna Samsonova, Moscow (RU); Veronika Aleksandrovna Kotliarova, Moscow (RU); Natalia Yurievna Rushkevich, Moscow (RU); Olga Sergeevna Beznoschenko, Stavropol territory (RU)

Correspondence Address: CERMAK NAKAJIMA LLP;ACS LLC, 127 S. Peyton Street, Suite 210, ALEXANDRIA, VA, 22314, US

(21) Appl. No.: 12/819,532

(22) Filed: Jun. 21, 2010

Related U.S. Application Data
(63) Continuation of application No. PCT/JP2008/073913, filed on Dec. 22, 2008.

(30) Foreign Application Priority Data
Dec. 21, 2007 (RU) 2007147438

(51) Int. Cl. C12P 13/06; C12N 1/21

(52) U.S. Cl. 435/116; 435/252.3; 435/252.32; 435/252.33

(57) ABSTRACT

A method for manufacturing (2S,3R,4S)-4-hydroxy-L-isoleucine or a salt thereof using an L-isoleucine-producing bacterium transformed with a DNA fragment containing a gene coding for a protein having L-isoleucine dioxygenase activity; and having the ability to produce (2S,3R,4S)-4-hydroxy-L-isoleucine.

This application is a continuation under 35 U.S.C. §120 of PCT Patent Application No. PCT/JP2008/073913, filed Dec. 22, 2008, which claims priority under 35 U.S.C. §119 to Russian Patent Application No. 2007147438, filed on Dec. 21, 2007, which are incorporated in their entireties by reference. The Sequence Listing in electronic format filed herewith is also hereby incorporated by reference in its entirety (File Name: 2010-06-21T_US-354_Seq_List; File Size: 7 KB; Date Created: Jun. 21, 2010).

### BACKGROUND OF THE INVENTION

1. Field of the Invention

The present invention relates to the microbiological industry, and specifically to a method for manufacturing 4-hydroxy-L-isoleucine, or a salt thereof, using an L-isoleucine producing bacterium. This bacterium is modified by the introduction of a DNA fragment which contains a gene coding for a protein having L-isoleucine dioxygenase activity, which results in production of (2S,3R,4S)-4-hydroxy-L-isoleucine.

2. Brief Description of the Related Art

4-Hydroxy-L-isoleucine is an amino acid which can be extracted and purified from fenugreek seeds (Trigonella foenum-graecum L. leguminosae). 4-hydroxy-L-isoleucine displays an insulinotropic activity, which is of great interest because its stimulating effect is clearly dependent on plasma glucose concentration in the medium, as demonstrated both in isolated perfused rat pancreas and human pancreatic islets (Sauvaire, Y. et al, Diabetes, 47: 206-210, (1998)). Such a glucose dependency has not been confirmed for sulfonylureas (Drucker, D. J., Diabetes 47: 159-169, (1998)), which is the only type of insulinotropic drug currently used to treat type II diabetes [or non-insulin-dependent diabetes (NIDD) mellitus (NIDDM)]. As a result, hypoglycemia remains a common undesirable side effect of sulfonylurea treatment (Jackson, J., and Bessler, R. Drugs, 22: 211-245; 295-320, (1981); Jennings, A. et al. Diabetes Care, 12: 203-208, (1989)). Improving glucose tolerance (Am. J. Physiol. Endocrinol., Vol. 287, E463-E471, 2004) has also been reported. This glucometabolism enhancement activity, and its potential application in pharmaceuticals and health foods, has been reported (Japanese Patent Application Laid-Open No. Hei 6-157302, US2007-000463A1).

4-hydroxy-L-isoleucine is only found in plants, and due to its particular insulinotropic action, might be considered as a novel secretagogue for the treatment of type II diabetes, a disease characterized by defective insulin secretion associated with various degrees of insulin resistance (Broca, C. et al, Am. J. Physiol. 277 (Endocrinol. Metab. 40): E617-E623, (1999)).

A method of oxidizing iron, ascorbic acid, 2-oxyglutaric acid, and oxygen-dependent isoleucine by utilizing the dioxygenase activity in fenugreek extract has been reported as a method for manufacturing 4-hydroxy-L-isoleucine (Phytochemistry, Vol. 44, No. 4, pp. 563-566, 1997). However, this method is insufficient to manufacture 4-hydroxy-L-isoleucine because the activity of the enzyme is inhibited by isoleucine concentrations of 20 Mm and above, the enzyme has not been identified, the enzyme is derived from plant extracts and cannot be obtained in sufficient quantities, and the enzyme is unstable.

An efficient eight-step synthesis of optically pure (2S,3R,4S)-4-hydroxyisoleucine with a 39% overall yield has been disclosed. The key steps of this synthesis involve the biotransformation of ethyl 2-methylacetoacetate to ethyl (2S,3S)-2-methyl-3-hydroxy-butanoate with Geotrichum candidum and an asymmetric Strecker synthesis (Wang, Q. et al, Eur. J. Org. Chem., 834-839 (2002)).

A short six-step chemoenzymatic synthesis of (2S,3R,4S)-4-hydroxyisoleucine with total control of stereochemistry, the last step being the enzymatic resolution by hydrolysis of a N-phenylacetyl lactone derivative using the commercially available penicillin acylase G immobilized on Eupergit C(E-PAC), has also been disclosed (Rolland-Fulcrand, V. et al, J. Org. Chem., 873-877 (2004)).

But currently, there have been no reports of producing (2S,3R,4S)-4-hydroxy-L-isoleucine by using a L-isoleucine producing bacterium modified by the introduction of a DNA fragment containing a gene coding for a protein having L-isoleucine dioxygenase activity.

### SUMMARY OF THE INVENTION

An aspect of present invention is to enhance production of (2S,3R,4S)-4-hydroxy-L-isoleucine (this term may include both the free form and a salt form thereof, and may also be referred to as “(2S,3R,4S)-4HIL”), to provide a method for manufacturing (2S,3R,4S)-4-hydroxy-L-isoleucine or a salt thereof by direct enzymatic hydroxylation of L-isoleucine. In this method, an L-isoleucine producing bacterium which is modified by the introduction of a DNA fragment containing a gene coding for a protein having L-isoleucine dioxygenase activity is employed.

A bacterium having a high level of L-isoleucine dioxygenase activity was isolated from nature, and a gene encoding L-isoleucine dioxygenase was cloned. It was found that L-isoleucine dioxygenase can be used in the synthesis of the (2S,3R,4S)-4-hydroxy-L-isoleucine.

The aspects of the present invention include providing a method for producing (2S,3R,4S)-4-hydroxy-L-isoleucine using an L-isoleucine producing bacterium modified by the introduction of a DNA fragment comprising a gene coding for a protein having L-isoleucine dioxygenase. The above aspect was achieved by finding that a bacterium with L-isoleucine dioxygenase activity produced (2S,3R,4S)-4-hydroxy-L-isoleucine.

It is an aspect of the present invention to provide a method for constructing the (2S,3R,4S)-4-hydroxy-L-isoleucine producing bacterium by introducing a DNA fragment comprising a gene coding for a protein having L-isoleucine dioxygenase activity into an L-isoleucine producing bacterium.

It is a further aspect of the present invention to provide the method as described above, wherein the bacterium is from a genus selected from the group consisting of Escherichia, Brevibacterium, Corynebacterium, Serratia, and Mycobacterium.

It is a further aspect of the present invention to provide the method as described above, wherein the bacterium is selected from the group consisting of Escherichia coli, Brevibacterium flavum, Corynebacterium glutamicum, Serratia marcescens, and Mycobacterium album.

It is a further aspect of the present invention to provide the method as described above, wherein the gene is selected from the group consisting of:

(a) a DNA comprising the nucleotide sequence of SEQ ID No: 1;

(b) a DNA that hybridizes under stringent conditions with a DNA having a nucleotide sequence complementary to the nucleotide sequence of SEQ ID No: 1 and encodes a protein having L-isoleucine dioxygenase activity;

(c) a DNA that encodes a protein comprising the amino acid sequence of SEQ ID No: 2;

(d) a DNA that encodes a protein having the amino acid sequence of SEQ ID NO: 2, except that one or several amino acid substitutions, deletions, insertions, additions, or inversions are present, and said protein has L-isoleucine dioxygenase activity; and

(e) a DNA that encodes a protein comprising an amino acid sequence that is at least 98% homologous to the amino acid sequence of SEQ ID NO: 2 and wherein said protein has L-isoleucine dioxygenase activity.

It is a further aspect of the present invention to provide a (2S,3R,4S)-4-hydroxy-L-isoleucine producing bacterium obtained by any of the above methods.

It is a further aspect of the present invention to provide a method for manufacturing (2S,3R,4S)-4-hydroxy-L-isoleucine or a salt thereof, comprising:
culturing a bacterium as described above in the culture medium; and
isolating (2S,3R,4S)-4-hydroxy-L-isoleucine.

It is a further aspect of the present invention to provide the method as described above, wherein the bacterium is modified to enhance the activity of the L-isoleucine dioxygenase.

It is a further aspect of the present invention to provide the method as described above, wherein the activity of the L-isoleucine dioxygenase is enhanced by increasing the expression of the gene encoding said L-isoleucine dioxygenase.

It is a further aspect of the present invention to provide the method as described above, wherein the expression of the L-isoleucine dioxygenase is increased by modifying an expression control sequence of the gene encoding the L-isoleucine dioxygenase or by increasing the copy number of the gene encoding the L-isoleucine dioxygenase.

It is a further aspect of the present invention to provide the method as described above, wherein the bacterium belongs to a genus selected from the group consisting of Escherichia, Brevibacterium, Corynebacterium, Serratia, and Mycobacterium.

It is a further aspect of the present invention to provide the method as described above, wherein the bacterium is selected from the group consisting of Escherichia coli, Brevibacterium flavum, Corynebacterium glutamicum, Serratia marcescens, and Mycobacterium album.

The present invention is described in detail below.

### BRIEF DESCRIPTION OF THE DRAWINGS

FIG. 1 shows the structure of the recombinant plasmid Pmw119-IDO(Lys, 23).

### DESCRIPTION OF THE PREFERRED EMBODIMENTS

### 1. Bacterium

The term “(2S,3R,4S)-4-hydroxy-L-isoleucine” or “(2S,3R,4S)-4HIL” or “4HIL” can refer to a single chemical compound, or a mixture of compounds containing (2S,3R,4S)-4-hydroxyisoleucine.

The term “bacterium” can include an enzyme-producing bacterium, a mutant or genetic recombinant of such bacterium in which the targeted enzymatic activity is present or has been enhanced, and the like.

L-isoleucine dioxygenase from microbial cells is hereinafter abbreviated as IDO.

Previously, the screening of environmental microorganisms revealed a unique microbe Bacillus thuringiensis strain 2-e-2, which could catalyze a reaction in which (2S,3R,45)-4HIL is directly formed from L-isoleucine (this term encompasses both the free form and a salt form thereof). The novel L-isoleucine dioxygenase was purified and isolated from the cultivated microbial cells, hereinafter abbreviated as IDO(Lys,23).

Furthermore, the N-terminal amino acid sequence of IDO(Lys,23) was determined by purifying dioxygenase derived from of Bacillus thuringiensis strain 2-e-2. Bacillus thuringiensis strain 2-e-2 was named Bacillus thuringiensis AJ110584 and deposited at the International Patent Organism Depositary, National Institute of Advanced Industrial Science and Technology (Central 6, 1-1, Higashi 1-chome, Tsukuba, Ibaraki 305-8566, Japan) on Sep. 27, 2006 and given an accession number of FERM BP-10688 under the provisions of Budapest Treaty.

The DNA encoding the IDO(Lys,23) that is identified in the Examples is shown in SEQ ID No: 1. Furthermore, the amino acid sequence of IDO(Lys,23) encoded by the nucleotide sequence of SEQ ID NO: 1 is shown in SEQ ID No: 2. SEQ ID NO: 2 is the amino acid sequence of IDO(Lys,23) encoded by the nucleotide sequence of SEQ ID NO: 1. IDO(Lys,23) of SEQ ID NO: 2 possesses the L-isoleucine dioxygenase activity, and catalyzes the reaction in which (2S,3R,45)-4HIL shown in the following formula (I) is directly synthesized from one molecule of L-isoleucine.

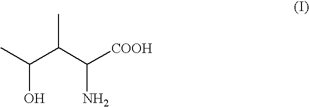

The DNA that encodes the IDO which catalyzes the reaction in which (2S,3R,4S)-4HIL is formed from L-isoleucine includes not only the DNA shown in SEQ ID No: 1. This is because there may be differences in the IDO nucleotide sequences among various species and strains of Bacillus which do not effect the activity of producing (2S,3R,45)-4HIL from L-isoleucine.

The DNA not only includes the isolated DNA encoding IDO, but also DNA sequences in which mutations have been artificially introduced, for example, a DNA that encodes IDO isolated from a chromosomal DNA of an IDO-producing microorganism as long as it encodes an IDO which is able to catalyze the desired reaction. Mutations may be artificially introduced using known methods such as by introducing site-specific mutations as described in Method. in Enzymol., 154 (1987).

DNA that hybridizes under stringent conditions with a DNA having a nucleotide sequence complementary to the nucleotide sequence of SEQ ID No: 1, and encodes a protein having IDO activity can also be used. The term “stringent conditions” can refer to those conditions under which a specific hybrid is formed and a non-specific hybrid is not formed. Although it is difficult to numerically express these conditions explicitly, by way of example, mention is made of those conditions under which DNA molecules having higher homology e.g. such as 70% or more, or in another example 80% or more, or in another example 90% or more, and in another example 95% or more homology, hybridize with each other, while DNA molecules having lower homology do not hybridize with each other, or those conditions under which hybridization occurs under usual washing conditions in Southern hybridization, that is, at a salt concentration of 0.1×SSC and 0.1% SDS at 37° C., or in another example 0.1×SSC and 0.1% SDS at 60° C., and in another example 0.1×SSC and 0.1% SDS at 65° C. The length of the probe may be suitably selected, depending on the hybridization conditions, and usually varies from 100 bp to 1 kbp. Furthermore, “L-isoleucine dioxygenase activity” typically indicates the synthesis of (2S,3R,45)-4HIL from L-isoleucine. However, when using a nucleotide sequence that hybridizes under stringent conditions with a nucleotide sequence complementary to the nucleotide sequence of SEQ ID No: 1, L-isoleucine dioxygenase activity of 10% or more, or in another example 30% or more, or in another example 50% or more, and still in another example 70% or more, can be retained for the protein having the amino acid sequence of SEQ ID No: 2 at 37° C. and pH 8.

Furthermore, the DNA encoding a protein which is substantially identical to the IDO encoded by the DNA of SEQ ID No: 1 can also be used. Namely, the following can be included:

(a) a DNA having the nucleotide sequence of SEQ ID No: 1;

(b) a DNA that hybridizes under stringent conditions with a DNA having a nucleotide sequence complementary to the nucleotide sequence of SEQ ID No: 1 and encodes a protein having the L-isoleucine dioxygenase activity;

(c) a DNA that encodes a protein having the amino acid sequence of SEQ ID No: 2;

(d) a DNA that encodes a protein having the amino acid sequence of SEQ ID NO: 2, except that one or several amino acid substitutions, deletions, insertions, additions, or inversions are present, and said protein having the L-isoleucine dioxygenase activity; and

(e) a DNA that encodes a protein having an amino acid sequence that is at least 70% homologous, or in another example at least 80% homologous, or in another example at least 90% homologous and still in another example at least 95% homologous to the amino acid sequence of SEQ ID NO:2 and wherein said protein has L-isoleucine dioxygenase activity.

Here, “one or several” can refer to the number of amino acid changes which does not significantly impair the 3D structure of the protein or the L-isoleucine dioxygenase activity, and more specifically, a number in the range of 1 to 78, or in another example 1 to 52, or in another example 1 to 26, and still in another example 1 to 13.

The substitution, deletion, insertion, addition or inversion of one or several amino acid residues should be conservative mutation(s) so that the activity is maintained. The representative conservative mutation is a conservative substitution. Examples of conservative substitutions include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val.

Furthermore, “L-isoleucine dioxygenase activity” can refer to the synthesis of (2S,3R,4S)-4HIL from L-isoleucine as described above. However, when the amino acid sequence of SEQ ID NO:2 contains a substitution, deletion, insertion, addition or inversion of one or several amino acid residues, L-isoleucine dioxygenase activity of 10% or more, or in another example 30% or more, or in another example 50% or more, and still in another example 70% or more, can be retained at 30° C. and pH 6.0. The L-isoleucine dioxygenase activity of the IDO can be measured by determination of (2S,3R,45)-4HIL formation by high-performance liquid chromatography (HPLC).

Furthermore, a homologue DNA of SEQ ID NO: 1 can be used as the gene encoding L-isoleucine dioxygenase. Whether the homologue DNA encodes L-isoleucine dioxygenase or not can be confirmed by measuring the L-isoleucine dioxygenase activity of the cell lysate of the microorganism in which the homologue DNA is overexpressed.

The homologue DNA of SEQ ID NO: 1 can also be prepared from the genome of another Bacillus species, for example, Bacillus cereus, and Bacillus weihenstephanensis.

The phrase “a bacterium belonging to the genus Escherichia” indicates a bacterium classified into the genus Escherichia according to the classification known to a person skilled in the art of microbiology. Examples of a bacterium belonging to the genus Escherichia include, but are not limited to, Escherichia coli (E. coli).

The bacterium belonging to the genus Escherichia is not particularly limited; however, e.g., bacteria described by Neidhardt, F. C. et al. (Escherichia coli and Salmonella typhimurium, American Society for Microbiology, Washington D.C., 1208, Table 1) can be used.

The phrase “a bacterium belonging to the genus Brevibacterium,” means that the bacterium is classified into the genus Brevibacterium according to the classification known to a person skilled in the art of microbiology. Examples of a bacterium belonging to the genus Brevibacterium include, but are not limited to, Brevibacterium flavum.

The phrase “a bacterium belonging to the genus Corynebacterium” means that the bacterium is classified into the genus Corynebacterium according to the classification known to a person skilled in the art of microbiology. Examples of a bacterium belonging to the genus Corynebacterium include, but are not limited to, Corynebacterium glutamicum.

The phrase “a bacterium belonging to the genus Serratia” means that the bacterium is classified into the genus Serratia according to the classification known to a person skilled in the art of microbiology. Examples of a bacterium belonging to the genus Serratia include, but are not limited to, Serratia marcescens.

The phrase “a bacterium belonging to the genus Mycobacterium” means that the bacterium is classified into the genus Mycobacterium according to the classification known to a person skilled in the art of microbiology. Examples of a bacterium belonging to the genus Mycobacterium include, but are not limited to, Mycobacterium album.

The term “L-isoleucine producing bacterium” can mean a bacterium which is able to produce and cause accumulation of L-isoleucine in a culture medium in an amount larger than a wild-type or parental strain, and can also mean that the microorganism is able to produce and cause accumulation in an amount of not less than 0.5 g/L, or in another example not less than 1.0 g/L of L-isoleucine.

Examples of the L-isoleucine producing bacterium can include, but are not limited to, mutants which are resistant to 6-dimethylaminopurine (JP 5-304969 A), mutants which are resistant to an isoleucine analogue such as thiaisoleucine and isoleucine hydroxamate, and mutants additionally which are resistant to DL-ethionine and/or arginine hydroxamate or the like (JP 5-130882 A).

In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

The L-isoleucine producing bacterium belonging to the genus Escherichia can be an Escherichia coli bacterium carrying the thrABC operon which includes the thrA gene coding for aspartokinase I-homoserine dehydrogenase I, and is not substantially inhibited by L-threonine. This bacterium also may contain the ilvGMEDA operon which includes the ilvA gene coding for threonine deaminase, is not substantially inhibited by L-isoleucine, and the region required for attenuation has been deleted. Furthermore, the host strain of said bacteria is defective in thrC gene, can proliferate in the presence of 5 mg/ml of L-threonine, is defective in threonine dehydrogenase activity, and the ilvA gene has a leaky mutation. Specific examples thereof include Escherichia coli strains AJ12919 and AJ13100 (U.S. Pat. No. 5,998,178) or the like.

The L-isoleucine producing bacterium belonging to the genus Escherichia can also be an Escherichia bacterium which contains the thrABC operon which includes the thrA gene coding for aspartokinase I-homoserine dehydrogenase I, and is not substantially inhibited by L-threonine. This bacterium may also contain the lysC gene coding for aspartokinase III and which is not substantially inhibited by L-lysine. Furthermore, this bacterium can contain the ilvGMEDA operon which includes the ilvA gene coding for threonine deaminase, which is not substantially inhibited by L-isoleucine, and the region required for the attenuation has been deleted (U.S. Pat. No. 5,998,178) or the like.

The bacterium belonging to the genus Escherichia can include the thrABC operon, the lysC gene and the ilvGMEDA operon, as described above, on a plasmid or plasmids on which they are loaded.

Furthermore, the L-isoleucine producing bacterium belonging to the genus Escherichia can be an Escherichia coli strain with enhanced phosphoenolpyruvate carboxylase and transhydrogenase activity, as well as enhanced aspartase activity (EP1179597 B1).

The L-isoleucine producing bacterium belonging to the genus Brevibacterium can be a Brevibacterium flavum or Brevibacterium lactofermentum bacterium. The bacterium can be densitized to both the feedback inhibition activity of acetohydroxy acid synthase and L-isoleucine inhibition of threonine deaminase. Specific examples thereof include Brevibacterium flavum strain AJ 12406 (FERM P-10143, FERM BP-2509) Brevibacterium lactofermentum AJ12403/pAJ220V3 (EP0356739 B1) and the like.

The L-isoleucine producing bacterium belonging to the corynebacteria can be a bacterium belonging to coryneform glutamic acid-forming mold, which is resistant to threoninehydroxamate. Specific examples thereof include Corynebacterium glutamicum strain H-4260 (JP62195293) and the like.

A DNA fragment containing the gene coding for L-isoleucine dioxygenase into the bacterium can be introduced by transformation of the bacterium with the vector containing the DNA fragment containing the gene coding for L-isoleucine dioxygenase. An exemplary vector, for example, can be a plasmid that is autonomously replicable in the cells of the chosen bacteria.

The phrase “[t]ransformation of a bacterium with DNA encoding a protein” can mean the introduction of the DNA into a bacterium, for example, by conventional methods. Transformation of this DNA will result in an increase in expression of the gene encoding the protein(s) as described herein, and will enhance the activity of the protein in the bacterial cell. Transformation can be accomplished by any known method that has previously been reported. For example, the treating of recipient cells with calcium chloride so as to increase permeability of the cells to DNA has been reported for Escherichia coli K-12 (Mandel, M. and Higa, A., J. Mol. Biol., 53, 159 (1970)), and may be used.

The presence or absence of the gene in the chromosome of the bacterium can be detected by well-known methods, including PCR, Southern blotting, and the like.

Preparing plasmid DNA, digestion and ligation of DNA, transformation, selection of an oligonucleotide as a primer, and the like, can be accomplished by ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

### 2. Method

The method can be a method for producing (2S,3R,4S)-4-hydroxy-L-isoleucine by cultivating the bacterium as described herein in a culture medium, and isolating the (2S,3R,4S)-4-hydroxy-L-isoleucine from the medium.

The chosen culture medium may be either synthetic or natural, so long as it includes a carbon source and a nitrogen source, minerals and, if necessary, appropriate amounts of nutrients which the bacterium may require for growth. The carbon source may include various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation can be performed under aerobic conditions, such as a shaking culture, and a stirring culture with aeration, at a temperature of 20 to 40° C., preferably 30 to 38° C. The pH of the culture can be between 5 and 9, or in another example between 6.5 and 7.2. The pH of the culture can be adjusted with ammonia, calcium carbonate, various acids, various bases, and buffers.

Separation and purification methods can be used in which the (2S,3R,45)-4HIL is contacted with an ion exchange resin to adsorb basic amino acids followed by elution and crystallization. Also, methods in which the product obtained by elution is discolored and filtrated with activated charcoal followed by crystallization to obtain (2S,3R,45)-4HIL can also be used.

### EXAMPLES

The present invention will be explained in further detail with reference to the following Examples; however, the invention is not limited thereto.

### Example 1

### Construction of the MG1655 (P_{Lac}-lacI-IlvA*)[pMW119-IDO(Lys, 23); pVIC40] Strain

1.1. Construction of the pMW119-IDO(Lys, 23) Plasmid.

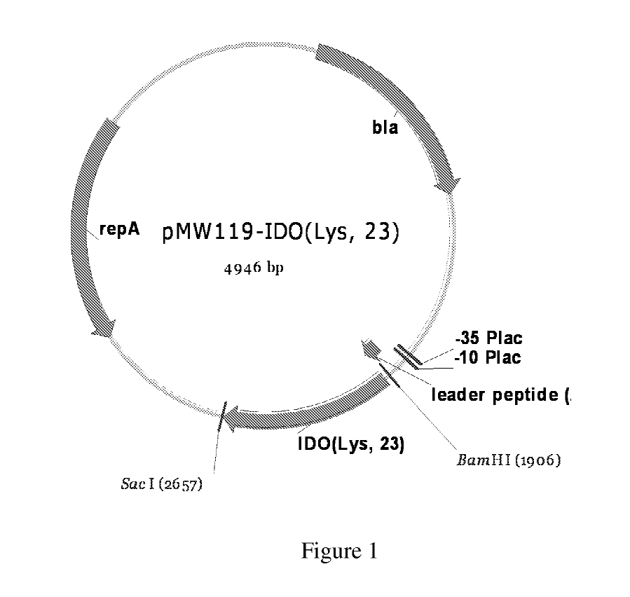

An 0.8 kb DNA fragment of the chromosome of the Bacillus thuringiensis strain 2-e-2 was amplified using oligonucleotides SVS 170 (SEQ ID No:3) and SVS 169 (SEQ ID No:4) as primers and purified chromosomal DNA as the template. The following PCR protocol was used: initial cycle for 30 seconds at 94° C.; 4 cycles for 40 seconds at 94° C.; 30 seconds at 49° C.; 40 seconds at 72° C.; 35 cycles for 30 seconds at 94° C.; 30 seconds at 54° C.; 30 seconds at 72° C. The resulting PCR fragment was digested with BamHI and SacI endonucleases and then ligated into the pMW119 vector which had been previously treated with the same endonucleases. Thus, the plasmid pMW119-IDO(Lys, 23) (FIG. 1) was constructed.

1.2. Construction of the MG1655 (P_{Lac}-lacI-IlvA*)[pMW119-IDO(Lys, 23); pVIC40] Strain.

Cells of the strain MG1655(P_{Lac}-lacI-IlvA*) (Sycheva E. V. et al., Biotechnologiya (RU), No. 4, 22-34, (2003)) were transformed with plasmid pMW119-IDO (Lys, 23). The resulting clones were selected on an X-gal/IPTG agar-plate (blue/white test). Thus, the strain MG1655(P_{Lac}-lacI-IlvA*) [pMW119-IDO(Lys, 23)] was obtained. The strain MG1655 (P_{Lac}-lacI-IlvA*) [pMW119-IDO(Lys, 23)] was transformed with plasmid pVIC40 (RU 1694643, U.S. Pat. No. 7,138,266). The resulting clones were selected on L-agar with Sm. Thus, the strain MG1655(P_{Lac}-lacI-IlvA*) [pMW119-IDO(Lys, 23), pVIC40] was obtained.

### Example 2

### Production of 4HIL by E. coli Strain MG1655(P_{Lac}-lacI-IlvA*) [pMW119-IDO(Lys, 23), pVIC40]

To test the effect of expression of the gene coding for IDO on 4HIL production, cells of the MG1655(P_{Lac}-lacI-IlvA*) [pMW119, pVIC40] and MG1655(P_{Lac}-lacI-IlvA*) [pMW119-IDO(Lys, 23), pVIC40] strains were inoculated into medium ILE [glucose—60 g/l, (NH₄)₂SO₄ 15 g/l, KH₂PO₄ 1.5 g/l, MgSO₄ 1 g/l, thiamin 0.001 g/l, Tryptone 1 g/l, Yeast extract 0.5 g/l, CaCO₃ 25 g/l, pH 7.0 (KOH), 1 mM IPTG, appropriate antibiotics (Ap, 100 mg/l; Sm, 100 mg/l)]. Cells were cultivated at 32° C. for 72 hours with vigorous agitation.

Then, the accumulation of Ile and 4HIL was investigated by HPLC-analysis. For HPLC analysis, a High pressure chromatograph (Waters, USA) with spectrofluorometer 1100 series (Agilent, USA) was used. The chosen detection wave range: excitation wavelength at 250 nm, range of emission wavelengths were 320-560 nm. The separation by accq-tag method was performed in a column Nova-Pak™ C18 150×3.9 mm, 4 μm (Waters, USA) at +40° C. Injection volume of the sample was 5 μl. The formation of amino acid derivatives and their separation was performed according to Waters manufacturer's recommendation (Liu, H. et al, J. Chromatogr. A, 828, 383-395 (1998); Waters accq-tag chemistry package. Instruction manual. Millipore Corporation, pp. 1-9 (1993)). To obtain amino acid derivatives with 6-aminoquinolyl-N-hydroxysuccinimidyl carbamate, the kit Accq-Fluor™ (Waters, USA) was used. The analysis by the accq-tag method was performed using concentrated Accq-tag Eluent A (Waters, USA). All solutions were prepared using Milli-Q water, standard solutions were stored at 4° C.

The results of measuring of the Ile and 4HIL produced by the MG1655(P_{Lac}-lacI-IlvA*) [pMW119, pVIC40] and MG1655(P_{Lac}-lacI-IlvA*) [pMW119-IDO(Lys, 23), pVIC40] strains are shown in Table 1. As follows from Table 1, MG1655(P_{Lac}-lacI-IlvA*) [pMW119-IDO(Lys, 23), pVIC40] produced 4HIL, as distinguished from MG1655(P_{Lac}-lacI-IlvA*) [pMW119, pVIC40].

TABLE 1

Resulted
conc., mM

| Strain | Ile | 4HIL |
|---|---|---|
| MG1655(P_{Lac}-lacI-IlvA*)[pMW119-IDO(Lys, 23); | 14 | 1 |
| pVIC40] | | |
| MG1655(P_{Lac}-lacI-IlvA*)[pMW119; pVIC40] | 15 | — |

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL APPLICABILITY

According to the present invention, production of (2S,3R,4S)-4-hydroxy-L-isoleucine, which is useful as a component of pharmaceutical compositions with insulinotropic activity, by a bacterium transformed with a DNA fragment containing a gene coding for a protein having L-isoleucine dioxygenase activity can be enhanced.

1. A method for producing a bacterium which is able to produce a (2S,3R,4S)-4-hydroxy-L-isoleucine, said method comprising introducing a DNA fragment comprising a gene coding for a protein having L-isoleucine dioxygenase activity into a bacterium which is able to produce L-isoleucine.

2. The method according to claim 1, wherein the bacterium belongs to a genus selected from the group consisting of Escherichia, Brevibacterium, Corynebacterium, Serratia, and Mycobacterium.

3. The method according to claim 1, wherein the bacterium is selected from the group consisting of Escherichia coli, Brevibacterium flavum, Corynebacterium glutamicum, Serratia marcescens, and Mycobacterium album.

4. The method according to claim 1, wherein the gene is selected from the group consisting of:
(a) a DNA comprising the nucleotide sequence of SEQ ID No: 1;
(b) a DNA that hybridizes under stringent conditions with a DNA having a nucleotide sequence complementary to the nucleotide sequence of SEQ ID No: 1 and encodes a protein having L-isoleucine dioxygenase activity;
(c) a DNA that encodes a protein comprising the amino acid sequence of SEQ ID No: 2;
(d) a DNA that encodes a protein having the amino acid sequence of SEQ ID NO: 2, except that one or several amino acid substitutions, deletions, insertions, additions or inversions are present, and said protein has L-isoleucine dioxygenase activity; and
(e) a DNA that encodes a protein comprising an amino acid sequence that is at least 98% homologous to the amino acid sequence of SEQ ID NO: 2 and wherein said protein has L-isoleucine dioxygenase activity.

5. A bacterium which is able to produce (2S,3R,4S)-4-hydroxy-L-isoleucine obtained by the method according to claim 1.

6. A method for manufacturing (2S,3R,4S)-4-hydroxy-L-isoleucine or a salt thereof, comprising:
culturing a bacterium according to claim 5 in the culture medium; and
isolating the (2S,3R,4S)-4-hydroxy-L-isoleucine.

7. The method according to claim 6, wherein the bacterium is modified to enhance the activity of the L-isoleucine dioxygenase.

8. The method according to claim 7, wherein the activity of the L-isoleucine dioxygenase is enhanced by increasing the expression of the gene encoding said L-isoleucine dioxygenase.

9. The method according to claim 8, wherein the expression of the L-isoleucine dioxygenase is increased by modifying an expression control sequence of the gene encoding the L-isoleucine dioxygenase or by increasing the copy number of the gene encoding the L-isoleucine dioxygenase.

10. The method according to claim 6, wherein the bacterium belongs to a genus selected from the group consisting of Escherichia, Brevibacterium, Corynebacterium, Serratia, and Mycobacterium.

11. The method according to claim 10, wherein the bacterium is selected from the group consisting of Escherichia coli, Brevibacterium flavum, Corynebacterium glutamicum, Serratia marcescens, and Mycobacterium album.


SEQUENCE LISTING (4 sequences)

SEQ ID NO: 1 - DNA, 717 bp, Bacillus thuringiensis strain FERM BP-10688
  CDS (1)..(717)
aaaatgagtggctttagcatagaagaaaaggtacatgaatttgaatctaaagggtttcttgaaatctcaaatgaaatctttttacaagaggaagagaatcatagtttattaacacaagcacagttagattattataatttggaagatgatgcgtacggtgaatgccgtgctagatcttattcaaggtatataaagtatgttgattcaccagattatattttagataatagtaatgattacttccaatctaaagaatataactatgatgatggcgggaaagttagacagttcaatagcataaatgatagctttttatgtaatcctttaattcaaaatatcgtgcgtttcgatactgagtttgcatttaaaacaaatataatagataaaagtaaagatttaattataggcttacatcaagtaagatataaagctactaaagaaagaccatcttttagttcacctatttggttacataaagatgatgaaccagtagtatttttacaccttatgaatttaagtaatacagctatcggcggagataatttaatagctaattctcctcgggaaattaatcagtttataagtttgaaggagcctttagaaactttagtatttggacaaaaggtcttccatgccgtaacgccacttggaacagaatgtagtacggaggcttttcgtgatattttattagtaacattttcttataaggagacaaaa

SEQ ID NO: 2 - protein, 239 aa, Bacillus thuringiensis strain FERM BP-10688
KMSGFSIEEKVHEFESKGFLEISNEIFLQEEENHSLLTQAQLDYYNLEDDAYGECRARSYSRYIKYVDSPDYILDNSNDYFQSKEYNYDDGGKVRQFNSINDSFLCNPLIQNIVRFDTEFAFKTNIIDKSKDLIIGLHQVRYKATKERPSFSSPIWLHKDDEPVVFLHLMNLSNTAIGGDNLIANSPREINQFISLKEPLETLVFGQKVFHAVTPLGTECSTEAFRDILLVTFSYKETK

SEQ ID NO: 3 - DNA, 65 bp, Artificial sequence
  primer SVS 170
ctctagaggatccttaagaaggagatataccatgaaaatgagtggctttagcatagaagaaaagg

SEQ ID NO: 4 - DNA, 35 bp, Artificial sequence
  primer SVS 169
gaattcgagctcttattttgtctccttataagaaa